(19) United States
(12) Patent Application Publication
(10) Pub. No.: US 2021/0298635 A1
(43) Pub. Date: Sep. 30, 2021

(54) SYSTEMS AND METHODS FOR SEDATION-LEVEL MONITORING

(71) Applicant: Covidien LP, Mansfield, MA (US)

(72) Inventors: Paul S. ADDISON, Edinburgh (GB); Cynthia A. Miller, Laguna Hills, CA (US)

(73) Assignee: Covidien LP, Mansfield, MA (US)

(21) Appl. No.: 17/178,138

(22) Filed: Feb. 17, 2021

Related U.S. Application Data
(60) Provisional application No. 63/000,161, filed on Mar. 26, 2020.

(51) Int. Cl. A61B 5/08; A61B 5/00

(52) U.S. Cl. CPC A61B 5/08; A61B 5/746; A61B 5/7405; A61B 5/0077; A61B 5/742

(57) ABSTRACT

Systems and methods are provided for monitoring the sedation level of patients. Images may be captured of a patient and regions of interest identified. Changes to image properties or to one or more regions of interest may be analyzed to generate physiological parameters for the patient. Physiological parameters may include information related to the patient's breathing behavior and/or activity. Physiological parameters may be used to determine a sedation level of the patient. If it is determined that the patient is over- or under-sedated, appropriate action may be taken, such as displaying an indication, activating an alarm, and/or causing an amount of sedative to be administered to the patient.

### CROSS-REFERENCE TO RELATED APPLICATIONS

This application claims the benefit of U.S. Provisional Application No. 63/000,161, filed Mar. 26, 2020, the complete disclosure of which is hereby incorporated herein by reference in its entirety.

### INTRODUCTION

Medical care providers often have reason to place patients in a state of sedation. To do so, providers often administer a sedative drug that with the intention of producing a state of calm or sleep. Often, providers will sedate patients in need of assistive mechanical ventilation. One benefit of sedation in this context is that sedation reduces the patient's physiological stress and increases the patient's tolerance to the ventilation, which may be invasive. When sedating a patient who is mechanically ventilated, it is useful to ensure that the level of sedation matches the physiological goals. For instance, if the goal is to reduce a patient's ventilatory drive, a deeper level of sedation is required. However, since this has the consequence of causing ventilator-induced, diaphragmatic dysfunction, it is usually important to ensure that sedation is light enough to allow the patient full control of breathing. Therefore it is important to ensure the patient is properly sedated.

### SUMMARY

Aspects of the present disclosure relate to utilization of non-contact monitoring to assess a patient's sedation level. For example, non-contact monitoring may include video-based patient monitoring. Video-based monitoring may include capturing images of a patient and analyzing those images over a period of time to assess the patient's level of sedation. For instance, multiple images of the patient may be captured from an image-capture device. From these images, physiological information about the patient may be determined. Physiological information about the patient, such as information about the patient's breathing or the patient's activity, may be related to the patient's sedation level. The physiological information may be used to determine the patient's sedation level and, in some examples, control administration of a sedative to the patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating a schematic diagram of an image-based monitoring system.

FIG. 2 is a block-diagram illustrating a computing device, server, and image-capture device.

FIG. 3 is an illustration of a schematic diagram that illustrates a patient and various patient regions of interest.

FIG. 4 is a diagram illustrating an example of a ventilator connected to a human patient.

FIG. 5 is a flowchart illustrating an example method for patient monitoring a physiological parameter of a patient

FIG. 6 is a flowchart illustrating an example method for monitoring a sedation level of a patient.

FIG. 7 is a flowchart illustrating an example method for monitoring a physiological parameter of a patient.

FIG. 8 is a flowchart illustrating an example method for monitoring a breathing parameter and an activity level of a patient.

FIG. 9 is a diagram illustrating an example display for physiological parameters and sedation level of a patient.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed above, sedation of a patient during ventilation is often desired, particularly when ventilation of the patient requires intubation. However, when too much sedative has been administered, a patient may become over-sedated. Over-sedation presents several risks to the patient. For example, over-sedation can lead to respiratory depression. Over time, continued over-sedation can lead to respiratory compromise and even respiratory failure. On the other hand, when an insufficient amount of sedative has been administered, a patient may be under-sedated. Under-sedation also presents risks to the patient. For example, under-sedation can lead to agitation, including restlessness, ventilator asynchrony, removal of tubes or catheters by the patient or, in extreme cases, combative or violent behavior of the patient. Proper sedation allows a patient on a ventilator to breathe as comfortably as possible while maintaining the ability to breathe spontaneously. In addition, proper management of sedation levels for intubated patients can shorten the necessary length of intubation and lead to earlier extubation.

Current methods for assessing sedation often rely on a care provider's periodic subjective assessment of the patient. For example, providers often use the Richmond Agitation-Sedation Score (RAS) or the Sedation-Agitation Scale (SAS), which include manual checklists or scoring. Among other problems, those assessment techniques may be deficient because they are subjective, intermittent, and require caregiver interaction. As such, scores may vary substantially from one care provider to another.

The present technology addresses such problems, among others, with systems and methods that are capable of objectively, continuously, and/or automatically monitoring a patient's sedation level. The monitoring helps ensure that the patient is properly sedated (e.g., neither over- nor under-sedated). For example, non-contact monitoring methods may be implemented to monitor activity levels and physiological parameters of the sedated patient. The non-contact monitoring methods may be based on the analysis of a video feed of the patient, such as described in in greater detail in U.S. patent application Ser. Nos. 16/219,360, 16/713,268, and 16/535,228, each of which is hereby incorporated by reference in its entirety. The activity levels and physiological parameters may then be utilized to determine the sedation level of the patient. In some examples, the determined sedation levels may be used to automatically control the amount of sedative that is provided to the patient.

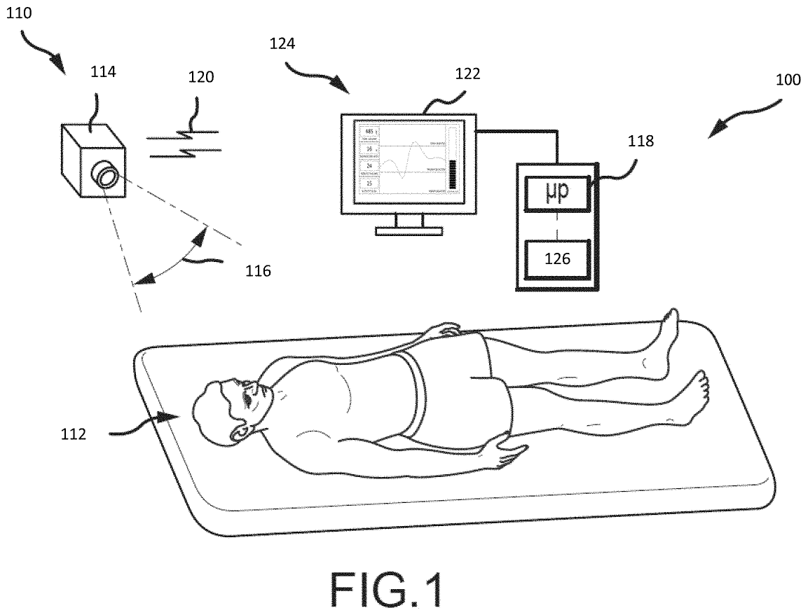

FIG. 1 is a schematic view of a video-based patient monitoring system 100 and a patient 112 according to an embodiment of the invention. The system 100 includes a non-contact detector 110 placed remote from the patient 112. In the depicted embodiment, the detector 110 includes a camera 114, such as a video camera. The camera 114 is remote from the patient, in that it is spaced apart from and does not contact the patient 112. The camera 114 includes a detector exposed to a field of view 116 that encompasses at least a portion of the patient 112.

The camera 114 generates a sequence of images over time. The camera 114 may be a depth sensing camera, such as a KINECT camera from Microsoft Corp. (Redmond, Wash.), a red-green-blue (RGB) camera, an infrared camera, a near-infrared camera, or any other type of image-capture device. A depth sensing camera can detect a distance between the camera and objects in its field of view. Such information may be used, as disclosed herein, to determine that a patient is within the field of view of the camera 114 and determine a region of interest (ROI) to monitor on the patient. Once an ROI is identified, that ROI can be monitored over time, and the change in depth of points within the ROI may represent movements of the patient associated with breathing. Accordingly, those movements, or changes of points within the ROI, may be used to determine a physiological parameter as disclosed herein.

In some embodiments, the system determines a skeleton outline of a patient to identify a point or points from which to extrapolate an ROI. For example, a skeleton outline may be used to find a center point of a chest, shoulder points, waist points, and/or any other points on a body. These points can be used to determine an ROI. For example, an ROI may be defined by filling in area around a center point of the chest. Certain determined points may define an outer edge of an ROI, such as shoulder points. In other embodiments, instead of using a skeleton outline, other points are used to establish an ROI. For example, a face may be recognized, and a chest area inferred in proportion and spatial relation to the face. In other embodiments described herein, the system may establish the ROI around a point based on which parts are within a certain depth range of the point. In other words, once a point is determined that an ROI should be developed from, the system can utilize the depth information from a depth sensing camera to fill out the ROI as disclosed herein. For example, if a point on the chest is selected, depth information is utilized to determine an ROI area around the determined point that is a similar distance from the depth sensing camera as the determined point. This area is likely to be a chest.

In another example, a patient may wear a specially configured piece of clothing that identifies points on the body such as shoulders or the center of the chest. A system may identify those points by identifying the indicating feature of the clothing. Such identifying features could be a visually encoded message (e.g., bar code, QR code, etc.), or a brightly colored shape that contrasts with the rest of the patient's clothing, etc. In some embodiments, a piece of clothing worn by the patient may have a grid or other identifiable pattern on it to aid in recognition of the patient and/or their movement. In some embodiments, the identifying feature may be stuck on the clothing using a fastening mechanism such as adhesive, a pin, etc. For example, a small sticker may be placed on a patient's shoulders and/or center of the chest that can be easily identified from an image captured by a camera. In some embodiments, the indicator may be a sensor that can transmit a light or other information to a camera that enables its location to be identified in an image so as to help define an ROI. Therefore, different methods can be used to identify the patient and define an ROI.

In some embodiments, the system may receive a user input to identify a starting point for defining an ROI. For example, an image may be reproduced on an interface, allowing a user of the interface to select a patient for monitoring (which may be helpful where multiple humans are in view of a camera) and/or allowing the user to select a point on the patient from which the ROI can be determined (such as a point on the chest). Other methods for identifying a patient, points on the patient, and defining an ROI may also be used, as described further below. In various embodiments, the ROI or portions of the ROI may be determined to move in accordance with respiratory patterns, to determine a physiological parameter of the patient, as described further below.

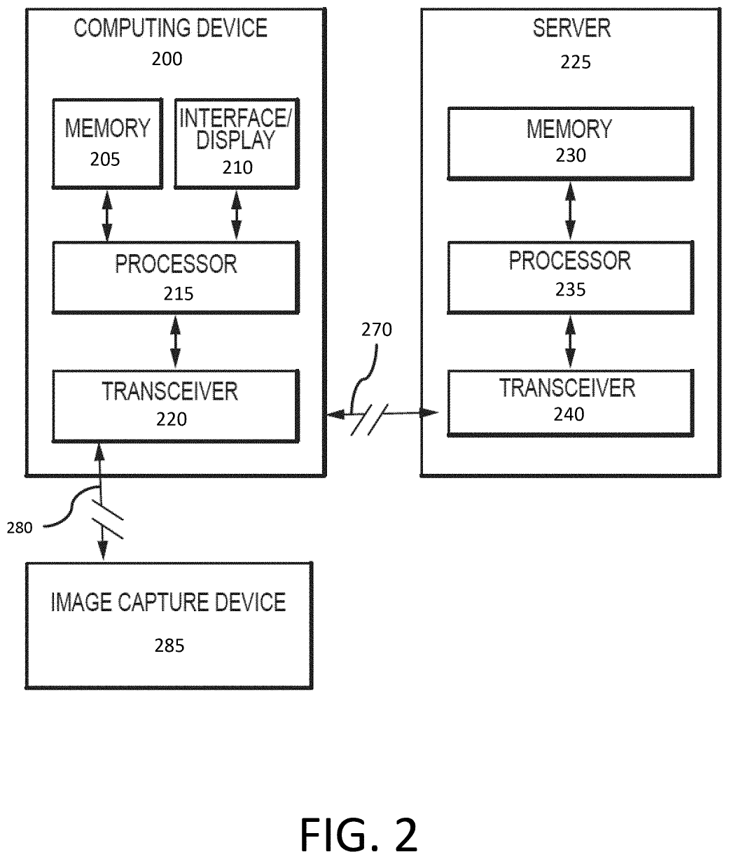

The detected images are sent to a computing device 124 through a wired or wireless connection 120. The computing device 124 includes a processor 118, a display 122, and hardware memory 126 for storing software and computer instructions. Sequential image frames of the patient are recorded by the video camera 114 and sent to the processor 118 for analysis. The display 122 may be remote from the camera 114, such as a video screen positioned separately from the processor and memory. Other embodiments of the computing device 124 may have different, fewer, or additional components than shown in FIG. 1. In some embodiments, the computing device 124 may be a server. In other embodiments, the computing device 124 of FIG. 1 may be additionally connected to a server (e.g., as shown in FIG. 2 and discussed below). The captured images/video can be processed or analyzed at the computing device 124 and/or a server to determine a physiological parameter of the patient 112 as disclosed herein.

FIG. 2 is a block diagram illustrating a computing device 200, a server 225, and an image-capture device 285 according to aspects of the present disclosure. In various examples, fewer, additional, and/or different components may be used in a system. The computing device 200 includes a processor 215 that is coupled to a memory 205. The processor 215 can store and recall data and applications in the memory 205, including applications that process information and send commands/signals according to any of the methods disclosed herein. The processor 215 may also display objects, applications, data, etc. on an interface/display 210. The processor 215 may also receive inputs through the interface/display 210. The processor 215 is also coupled to a transceiver 220. With this configuration, the processor 215, and subsequently the computing device 200, can communicate with other devices, such as the server 225 through a connection 270 and the image-capture device 285 through a connection 280. For example, the computing device 200 may send to the server 225 information determined about a patient from images captured by the image-capture device 285 (such as a camera), such as depth information of a patient in an image or physiological parameter information determined about the patient, as disclosed herein. The computing device 200 may be the computing device of FIG. 1.

Accordingly, the computing device 200 may be located remotely from the image-capture device 285, or the computing device may be local and close to the image-capture device 285 (e.g., in the same room). In various embodiments disclosed herein, the processor 215 of the computing device 200 may perform the steps disclosed herein. In other embodiments, the steps may be performed on a processor 235 of the server 225. In some embodiments, the various steps and methods disclosed herein may be performed by both of the processors 215 and 235. In some embodiments, certain steps may be performed by the processor 215 while others are performed by the processor 235. In some embodiments, information determined by the processor 215 may be sent to the server 225 for storage and/or further processing.

In some embodiments, the image-capture device 285 is a remote sensing device such as a video camera. In some embodiments, the image-capture device 285 may be some other type of device, such as a proximity sensor or proximity sensor array, a heat or infrared sensor/camera, a sound/acoustic or radiowave emitter/detector, or any other device that may be used to monitor the location of a patient and an ROI of a patient to determine a physiological parameter. Body imaging technology may also be utilized to generate physiological parameters according to the methods disclosed herein. For example, backscatter x-ray or millimeter wave scanning technology may be utilized to scan a patient, which can be used to define an ROI and monitor movement for physiological parameter calculations. Advantageously, such technologies may be able to “see” through clothing, bedding, or other materials while giving an accurate representation of the patient's skin. This may allow for more accurate physiological parameter measurements, particularly if the patient is wearing baggy clothing or is under bedding. The image-capture device 285 can be described as local because the device is relatively close in proximity to a patient so that at least a part of a patient is within the field of view of the image-capture device 285. In some embodiments, the image-capture device 285 can be adjustable to ensure that the patient is captured in the field of view. For example, the image-capture device 285 may be physically movable, may have a changeable orientation (such as by rotating or panning), and/or may be capable of changing a focus, zoom, or other characteristic to allow the image-capture device 285 to adequately capture a patient for monitoring. In various embodiments, after an ROI is determined, a camera may focus on the ROI, zoom in on the ROI, center the ROI within a field of view by moving the camera, or otherwise may be adjusted to allow for better and/or more accurate tracking/measurement of the movement of a determined ROI.

The server 225 includes a processor 235 that is coupled to a memory 230. The processor 235 can store and recall data and applications in the memory 230. The processor 235 is also coupled to a transceiver 240. With this configuration, the processor 235, and subsequently the server 225, can communicate with other devices, such as the computing device 200 through the connection 270.

The devices shown in the illustrative embodiment may be utilized in various ways. For example, any of the connections 270 and 280 may be varied. Any of the connections 270 and 280 may be a hard-wired connection. A hard-wired connection may involve connecting the devices through a USB (universal serial bus) port, serial port, parallel port, or other type of wired connection that can facilitate the transfer of data and information between a processor of a device and a second processor of a second device. In another embodiment, any of the connections 270 and 280 may be a dock where one device may plug into another device. In other embodiments, any of the connections 270 and 280 may be a wireless connection. These connections may take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another embodiment, the various devices may connect through an internet (or other network) connection. That is, any of the connections 270 and 280 may represent several different computing devices and network components that allow the various devices to communicate through the internet, either through a hard-wired or wireless connection. Any of the connections 270 and 280 may also be a combination of several modes of connection.

The configuration of the devices in FIG. 2 is merely one physical system on which the disclosed embodiments may be executed. Other configurations of the devices shown may exist to practice the disclosed embodiments. Further, configurations of additional or fewer devices than the ones shown in FIG. 2 may exist to practice the disclosed embodiments. Additionally, the devices shown in FIG. 2 may be combined to allow for fewer devices than shown or separated such that more than the three devices exist in a system. It will be appreciated that many various combinations of computing devices may execute the methods and systems disclosed herein. Examples of such computing devices may include other types of medical devices and sensors, infrared cameras/detectors, night vision cameras/detectors, other types of cameras, radio frequency transmitters/receivers, smart phones, personal computers, servers, laptop computers, tablets, blackberries, RFID enabled devices, or any combinations of such devices.

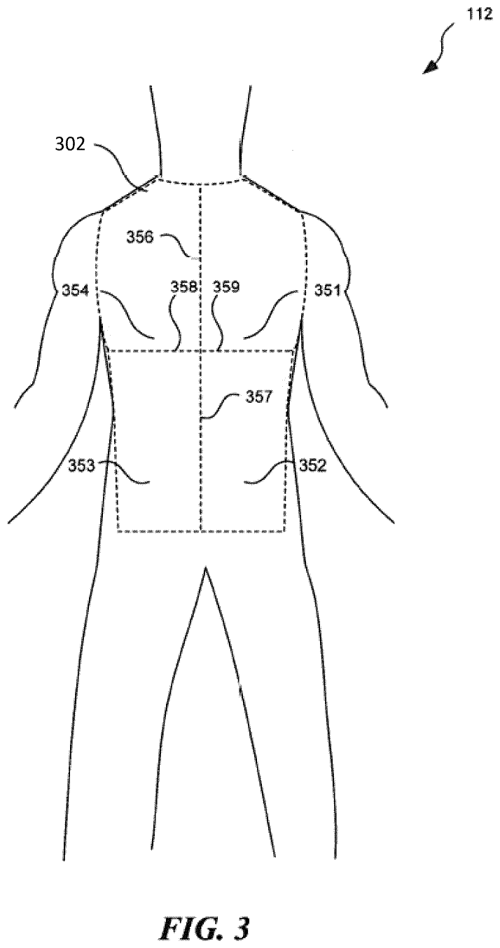

FIG. 3 is a schematic view of a patient 112 showing various regions of interest (ROIs) that can be defined by video-based patient monitoring systems configured in accordance with various embodiments of the present technology. As discussed above, a video-based patient monitoring system can define a ROI using a variety of methods (e.g., using extrapolation from a point on the patient 112, using inferred positioning from proportional and/or spatial relationships with the patient's face, using parts of the patient 112 having similar depths from the camera 114 as a point, using one or more features on the patient's clothing, using user input, etc.). In some embodiments, the video-based patient monitoring system may define an aggregate ROI 302 that includes both sides of the patient's chest as well as both sides of the patient's abdomen. As discussed in greater detail below, the aggregate ROI 302 can be useful in determining a patient's aggregate tidal volume, minute volume, and/or respiratory rate, among other aggregate breathing parameters. In these and other embodiments, the system 100 can define one or more smaller regions of interest within the patient's torso. For example, the system 100 can define ROI's 351-354. As shown, ROI 351 corresponds to the left half of the patient's chest, ROI 352 corresponds to the left half of the patient's abdomen, ROI 353 corresponds to the right half of the patient's abdomen, and ROI 354 corresponds to the right half of the patient's chest. In these and other embodiments, the system 100 can define other regions of interest in addition to or in lieu of the ROI's 302, 351, 352, 353, and/or 354. For example, the system 100 can define a ROI 356 corresponding to the patient's chest (e.g., the ROI 351 plus the ROI 354) and/or a ROI 357 corresponding to the patient's abdomen (e.g., the ROI 352 plus the ROI 353).

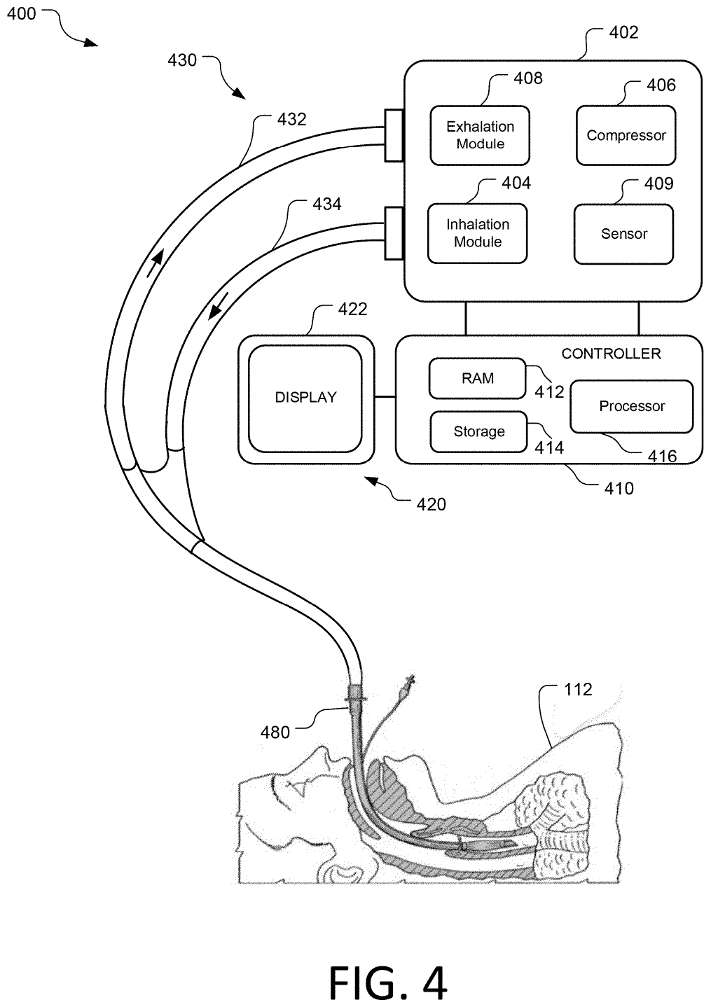

FIG. 4 is a diagram illustrating an example of a ventilator 400 connected to a patient 112. Ventilator 400 includes a pneumatic system 402 (also referred to as a pressure-generating system 402) for circulating breathing gases to and from patient 112 via the ventilation tubing system 430, which couples the patient to the pneumatic system via an invasive (e.g., endotracheal tube, as shown) or a non-invasive (e.g., nasal mask) patient interface.

Ventilation tubing system 430 may be a two-limb (shown) or a one-limb circuit for carrying gases to and from the patient 112. In a two-limb example, a fitting, typically referred to as a “wye-fitting” 470, may be provided to couple a patient interface 480 to an inhalation limb 434 and an exhalation limb 432 of the ventilation tubing system 430.

Pneumatic system 402 may have a variety of configurations. In the present example, system 402 includes an exhalation module 408 coupled with the exhalation limb 432 and an inhalation module 404 coupled with the inhalation limb 434. Compressor 406 or other source(s) of pressurized gases (e.g., air, oxygen, and/or helium) is coupled with inhalation module 404 to provide a gas source for ventilatory support via inhalation limb 434. The pneumatic system 402 may include a variety of other components, including mixing modules, valves, sensors, tubing, accumulators, filters, etc. The ventilator may include a sensor 409. Sensor 409 may be configured to measure and collect a variety of patient information. In an example, the ventilator sensor 409 is configured to monitor pressure and flow of gas at a point in the ventilation circuit. In other examples, there is more than one sensor, and each sensor is configured to monitor pressure and flow at different points in the ventilation circuit. For example, pressure and flow sensors may be placed so as to model the patient's breathing behavior and optimize the ventilator's operation to provide safe and effective ventilation.

Controller 410 is operatively coupled with pneumatic system 402, signal measurement and acquisition systems, and an operator interface 420 that may enable an operator to interact with the ventilator 400 (e.g., change ventilator settings, select operational modes, view monitored parameters, etc.). Controller 410 may include memory 412, one or more processors 416, storage 414, and/or other components of the type found in command and control computing devices. In the depicted example, operator interface 420 includes a display 422 that may be touch-sensitive and/or voice-activated, enabling the display 422 to serve both as an input and output device.

The memory 412 includes non-transitory, computer-readable storage media that stores software that is executed by the processor 416 and which controls the operation of the ventilator 400. In an example, the memory 412 includes one or more solid-state storage devices such as flash memory chips. In an alternative example, the memory 412 may be mass storage connected to the processor 416 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 416. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems (e.g. patient monitoring system 100), may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

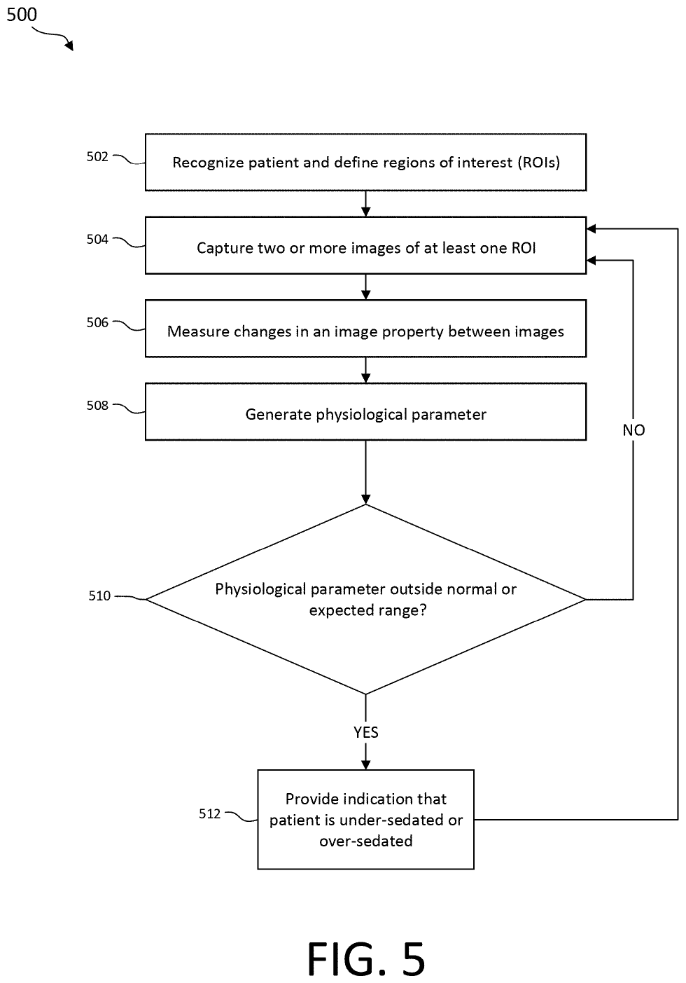

FIG. 5 illustrates an example method 500 for patient monitoring. All or a subset of the steps of method 500 may be executed by various components of a non-contact patient-monitoring system, such as a local computing device (e.g., computing device 200), a server (e.g., server 225), another remote device, a display (e.g., display 422), an image-capture device (e.g., camera 114), and/or other devices described herein. The method begins at step 502 where a patient is recognized and at least one region of interest (ROI) is defined. Step 502 can be performed by an image-capture device (e.g., image-capture device 285), a computing device (e.g., computing device 200), and/or a server (e.g., server 225). In an example, a patient is recognized by identifying the patient using facial-recognition hardware and/or software. An ROI can be defined according to the descriptions above with respect to FIGS. 1 through 3. For example, defining an ROI may include extrapolating from a point on the patient, using inferred positioning from proportional and/or spatial relationships with the patient's face, using parts of the patient having similar depths from a camera (e.g., camera 114), using features from the patient's clothing, etc. In another example, a manual selection (e.g., a selection at computing device 200) may be received from a care provider interacting with a computing device to select or define a patient and an ROI.

At operation 504, two or more images of at least one ROI are captured. Operation 504 can be performed by an image-capture device (e.g., image-capture device 285). In an example, two or more images can be captured by capturing a video sequence of the patient. In other examples, two or more images can be captured by capturing separate still images of the patient. The captured images may include the entire patient or may only include the defined ROI. In examples where the captured images include the entire patient, the captured images may later be processed to isolate or focus on a defined ROI.

At operation 506, changes between the two or more images are measured or otherwise determined. Operation 506 can be performed by an image-capture device (e.g., image-capture device 285), a computing device (e.g., computing device 200), and/or a server (e.g., server 225). In an example, the two or more images have at least one image property, such as temperature, color, movement, or any other property that can be measured by the image-capture device used to capture the two or more images. In some examples, the images may be captured by a depth-sensing camera, and the images may contain an indication of the image depth at various positions within the image. In such an example, image depth may be an image property of the two or more images. At operation 506, the image property is measured or determined in each of the two or more images and the change, if any, between the images is determined. As an example, a depth-sensing camera may capture two images with image depth as an image property. At operation 506, the difference in image depth for a defined ROI may be determined.

At operation 508, a physiological parameter is generated for the patient based on the measured change, or lack thereof, in the image property. Operation 508 may be performed by a computing device (e.g., computing device 200) and/or a server (e.g., server 225). In an example, the physiological parameter may be related to the patient's breathing (i.e., a breathing parameter). In other examples, the physiological parameter may be related to the patient's activity level (i.e., an activity parameter). A breathing parameter may be a measurement of, among other things, the patient's tidal volume, minute volume, respiratory rate, the ratio of breathing frequency divided by tidal volume, spontaneous inspiratory time, apnea, inspiratory time to expiratory time ratio, and/or any combination of the above. In an example, a breathing parameter (e.g., tidal volume) is generated by monitoring the movement of a patient's chest as the patient inhales and exhales. In such an example, the patient's chest may be defined as an ROI. The movement of the ROI may be monitored by, for example, capturing images with a depth-sensing camera. Vectors may be associated with points within the defined ROI and changes to the vectors may be related to changes in an image property (e.g., image depth) over time. The changes to the vectors may correspond to changes in the size of the patient's chest and the changes may be further analyzed (e.g., by integration or other appropriate analytical techniques) to generate a breathing parameter relevant to the patient's breathing behavior.

Similar techniques may be used to generate an activity parameter. In examples, an activity parameter may be, among other things, a measurement of an amount of patient movement, a type of patient movement, a frequency of patient movement, a rapidity of patient movement, a heart rate of the patient, paradoxical breathing, response to sound, response to physical stimulation, eye movement of the patient, or any combination of the above. An activity parameter may be generated based on changes in the captured images. For instance, a patient ROI may be defined to include the patient's head and neck area. In an example, changes to the head-and-neck ROI in the captured images may be measured to determine a patient activity parameter. As an example, frequent movement measured in the head-and-neck ROI may be identified as indicating patient agitation. In such an example an activity parameter may be generated to indicate the amount, type, frequency, etc. of the detected movement in the defined ROI. In another example, changes to the neck ROI in the captured images may be analyzed to determine a patient's muscle activity. In an example, a patient's muscle activity is used to determine the effort the patient is exerting in breathing. If a patient is having difficulty breathing, it may be determined through analyzing a patient's neck ROI or any other ROI and observing muscle recruitment. As used herein, muscle recruitment refers to the activation of additional motor units (e.g., muscles) to a given motor task (e.g., breathing). An activity parameter may include an indication of muscle recruitment and/or an indication of whether the patient is experiencing difficulty breathing.

At operation 510, a determination is made regarding whether the generated physiological parameter falls outside of a normal or expected range for that physiological parameter. This determination may be made by comparing the generated physiological parameter to one or more parameter thresholds. In an example, there is an upper threshold and a lower threshold for a generated physiological parameter. The normal or expected range for the physiological parameter falls between the lower and upper thresholds. Thus, if the physiological parameter exceeds the upper threshold or falls below the lower threshold, a determination may be made that the physiological parameter is outside the normal or expected range for that parameter. The normal or expected range for the physiological parameter may be based on a clinical data from a population of different patients. The normal or expected range may be dependent on the physical characteristics of the patient, such as the patient's age, height, weight, gender, etc. The range may be dependent on the patient's health condition or treatment status, such as whether the patient is suffering from an illness or whether the patient has been sedated and/or for how long the patient has been sedated.

The normal or expected range for the physiological parameter may also be based on a prior history of the patient being monitored. For instance, physiological parameters for the patient being monitored may have been recorded prior to the patient undergoing sedation or receiving a sedative. Those recorded physiological parameters may be analyzed to determine what is normal or expected for that patient. As an example, the average recorded value for a particular physiological parameter may be used as the expected or normal value of for the physiological parameter. The upper and lower thresholds may be based on tolerance, such as plus or minus 10% or plus or minus 20%, among other tolerances. The upper and lower thresholds may also be based on the statistical distributions of the recorded values for the physiological parameters of the patient. For instance, the upper threshold maybe one standard deviation above the average recorded value and the lower threshold may be one standard deviation below the average recorded value.

As an example, the physiological parameter may be a breathing parameter and may be a patient's respiratory rate. In such an example, there may be a normal or expected range of respiratory rates for a patient. That normal range for the respiratory rate may be based on clinical data of a population of people having characteristics similar to the monitored patient, such as similar height, weight, age, gender, etc. In other examples, the normal respiratory rate may be determined from the monitored patient's respiratory rate prior to being sedated. Such measurements of the patient's respiratory rate may occur immediately before the sedation or may occur in prior appointments or visits with a medical care provider.

Regardless of how the normal range is determined, the comparison between the physiological parameter (e.g., respiratory rate) and the normal or expected range may be used to determine the patient's sedation level. For instance, the patient's respiratory rate may be affected by the patient's level of sedation. As an example, a decrease in a patient's respiratory rate may be indicative of an increase in sedation level. In contrast, an increase in a patient's respiratory rate may be indicative of a decrease in sedation level. Accordingly, when the patient's respiratory rate approaches or passes an upper or lower threshold, these changes to the respiratory rate may be an indication that the patient is over- or under-sedated.

Thus, if the determination at operation 510 is “YES” (i.e., the physiological parameter is outside the normal or expected range), the method 500 proceeds to operation 512, where an indication is provided that the patient is over- or under-sedated. After the indication is provided in operation 510, or if the determination at operation 510 is “NO” (i.e., the physiological parameter is not outside the normal or expected range), the method 500 returns to operation 504, where the method 500 continues to capture images of the patient and continues to generate a physiological parameter for the patient. In this way, the method advantageously achieves continuous or substantially continuous monitoring of the patient, such that any deviation from the normal or expected range for a physiological parameter may be quickly detected and an indication quickly provided.

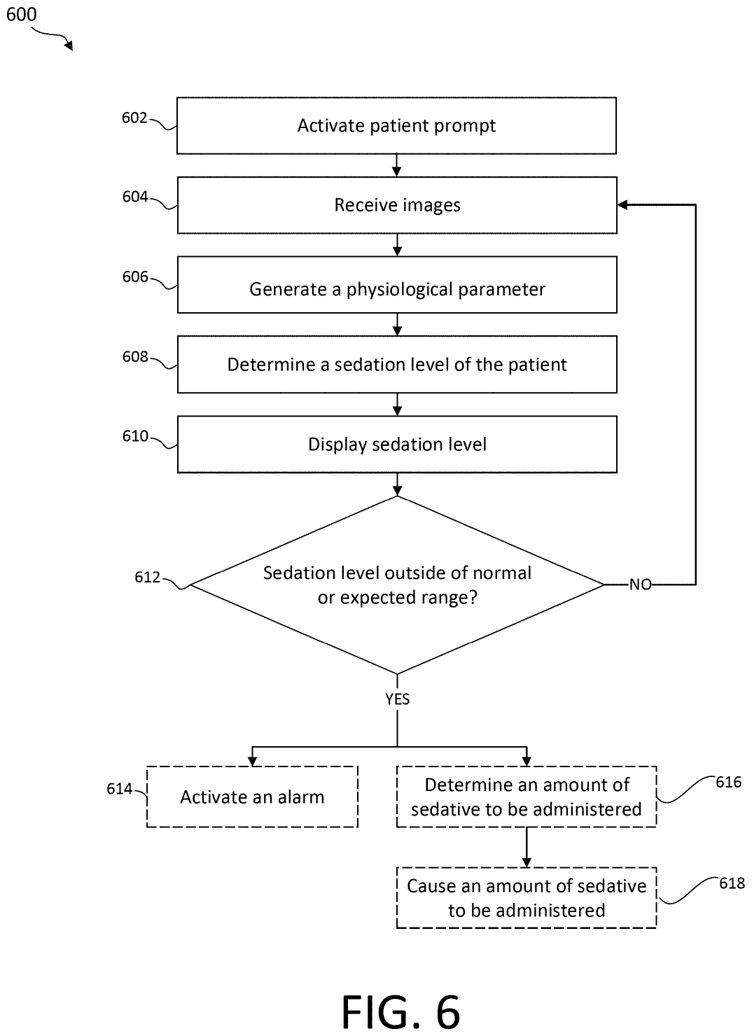

FIG. 6 illustrates an example method 600 for patient monitoring. All or a subset of the steps of method 600 may be executed by various components of a non-contact patient monitoring system, such as a local computing device (e.g., computing device 200), a server (e.g., server 225), another remote device, a display (e.g., display 422), an image-capture device (e.g., camera 114), and/or other devices described herein. At operation 602 of method 600, a patient prompt may be activated. In examples, a patient prompt is an auditory, visual, haptic, or other stimulus provided to a patient. In such an instance, a computing device may include or may be communicatively linked to a speaker, a display, and/or other mechanical elements, such as haptics, motors, or other elements capable of physically engaging the patient. The computing device or an associated server may send a signal to the speaker and/or display in order to activate a patient prompt. In other examples, a prompt to a caregiver to provide a stimulus may be generated. The prompt may indicate that a caregiver should provide a certain type of stimulus, such as an auditory or physical stimulus. Certain physiological parameters, such as activity parameters, may relate to a patient's response to a stimulus. For example, an activity parameter may be a measurement of patient movement in response to an auditory stimulus. An example auditory stimulus could be an auditory recitation of the patient's name. Auditory stimulus, and any other patient prompt, may have various degrees of stimulation. For example, there may be various levels of loudness of an auditory stimulus or varying levels of brightness to a visual stimulus. The brightness and loudness levels may be recorded along with the physiological parameters recorded with the patient's response to help ensure objective or consistent criteria are used. In examples, more than one patient prompt may be activated. For instance, a patient prompt that is an auditory stimulus may begin at a lower level of loudness and increase in loudness as the prompt is repeated. In this way, the images may capture the patient's level of alertness by determining the level of loudness necessary to invoke a response from the patient.

At operation 604, images are received. Operation 604 can be performed by a computing device (e.g., computing device 200) and/or a server (e.g., server 225). In examples where the method performs operation 602, the received images may include images captured before, during, and after the activation of the patient prompt, so as to capture the patient's response, if any, to the patient prompt. For instance, the images may be captured for a set time period subsequent to the initiation of that patient prompt in operation 602. The duration of the set time period may be different for each type of prompt or for each type of physiological parameter being measured. In other examples, the images are captured in a substantially continuous manner before, during, and after the patient prompt is initiated. As discussed herein, images may be captured by any suitable image-capture device (e.g., image-capture device 285). Images may include one or more image properties, such as image depth, image color, temperature, brightness, or any other property detectable by any image-capture device.

At operation 606, a physiological parameter is generated based on the images received in operation 604. As discussed herein, the physiological parameter may relate to a patient's breathing behavior, a patient's activity level, or any other physiological behavior that may be relevant to determining a patient's level of sedation. The physiological parameter may be generated based on analysis of the received images. Analysis of the images may include measurement of image properties and measurement of changes in image properties over time, as described herein such as with respect to FIGS. 1-4. In an example, the received images are captured by a depth-sensing camera and include image depth information. The image may further be defined by one or more ROIs, as described herein. In examples where operation 602 is performed to activate a patient prompt, a physiological parameter may be generated based on the received images as well as the images' relationship to the timing of the patient prompt. For instance, the received images may each include a time stamp or may be otherwise associated with a point in time. Based on the activation of the patient prompt, the images may be categorized or otherwise identified according to their temporal relationship to the activation of the patient prompt. For example, the received images may be identified as being captured before, during, or after the patient prompt. In this way, a physiological parameter may be generated based on the images' relationship to the patient prompt. For example, if changes are detected in the received images that have been identified as having been captured after the patient prompt, a physiological parameter may be generated that relates to the patient's level of arousal or arousability.

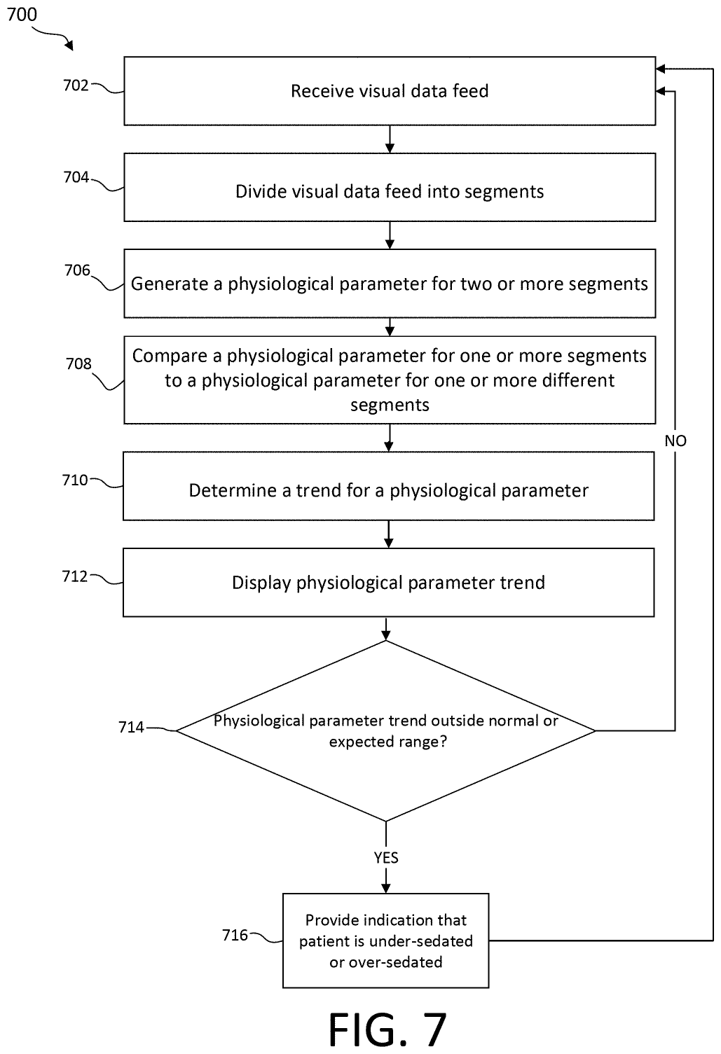

At operation 608, a sedation level of the patient is determined based on at least the physiological parameter generated in operation 606. Determining a sedation level may involve analyzing one or more physiological parameters generated in operation 608. For example, determining a sedation level may include comparing one or more generated physiological parameters to one or more parameter thresholds. In another example, determining the sedation level includes determining a rate of change of one or more physiological parameters. The rate of change of the one or more physiological parameters may similarly be compared to one or more rate-of-change thresholds, as described in greater detail with reference to FIG. 7, below. The physiological parameters and the rate at which they are changing may provide an indication of how a patient is being affected by the sedatives that have been administered.

As an example, physiological parameters of tidal volume, minute volume, and respiratory rate are all physiological parameters that relate to the patient's breathing behavior. A patient's breathing behavior is affected by administration of a sedative. In some examples, a patient breathes more deeply (i.e., a larger tidal volume) and more slowly (i.e., a lower respiratory rate) when under the influence of sedatives. In other examples, a patient may breathe less deeply and more rapidly. In some examples, a reduced minute volume may be indicative of over sedation. In some cases, sedation may cause respiratory suppression and therefore the patient may breathe more shallowly and also experience periods of apnea (e.g., periodic breathing). The respiratory suppression may cause diaphragm dysfunction from lack of use. In other cases, if a patient is intentionally sedated to suppress respiratory drive, which may be done in acute phases of acute respiratory distress syndrome (ARDS), then under sedation may cause the patient to attempt to control ventilation. Such as scenario may result in asynchrony, which may also damage the diaphragm and the lungs. In some examples, the measured tidal volume measurement may be compared to ARDS net guidelines of 4-6 mL/kg to determine adherence to the guidelines. Thus, the extent to which these breathing parameters deviate from the patient's normal or expected breathing behavior is an indication of the extent to which the sedatives are affecting the patient. Similarly, a patient's activity is often affected by administration of a sedative. For example, when a patient is sedated, the amount and/or frequency of patient movement often decrease. Thus, the extent to which these activity parameters deviate from the patient's normal or expected activity may be an indication of the extent to which the sedatives are affecting the patient. In other examples, changes (and the rate of such changes) to physiological parameters may be indicative of the extent to which a sedative is affecting a patient.

In other examples, determining a sedation level of the patient involves analysis and combination of two or more generated physiological parameters, such as the physiological parameters generated in operation 606. In an example, generated physiological parameters may be normalized based on comparison to a normal or expected parameter level or value. For example, the two physiological parameters may be a breathing parameter and a tidal volume. The generated tidal volume may be compared to an expected tidal volume for that patient. From the comparison, a normalized value may be derived. For instance, a range of tidal volume values may be normalized from 0 to 100, where 50 represents a normal tidal volume for a patient. The normalized values may be unitless. The normalization range may be patient-specific or may be common to all patients. Accordingly, each generated physiological parameter (whether a breathing parameter, an activity parameter, or any other parameter) may be compared to normal or expected values, and normalized values may be generated as a result.

The normalization may further be based on known, derived, or predicted relationships between a specific physiological parameter and sedation. For example, a physiological parameter may be useful in the sedation-level determination if (a) there is a relationship between the parameter and sedation and (b) some aspect of that relationship is known and incorporated into the sedation-level determination. Some of these relationships are already known or have already been theorized. Other relationships may be discovered based on clinical research, theoretical analysis, observation, etc. As an example, a higher respiratory rate corresponds to a lower sedation level. Likewise, a decreased tidal volume and/or a reduced minute volume corresponds to a greater sedation level. Certain activity parameters, such as movement frequency, also correspond to a lower sedation level. It will be appreciated that there are many other known relationships between physiological parameters and sedation levels. In examples where the relationship is known, the correlation may be used to normalize the physiological parameter. For instance, if the physiological parameter has a negative correlation with sedation level (e.g., respiratory rate), the normalization values may be negative so that increases in respiratory rate lead to a normalized value that is more negative. In this example, when the normalized respiratory rate is combined to generate a sedation level, the more negative normalized value will result in a lower sedation level. It will be appreciated that any number of relationships and/or correlations may be factored into the normalization and/or into the determination of sedation levels.

Once normalized values have been generated for each physiological parameter, the normalized values may be combined to determine a sedation level. In an example, the normalized physiological parameters are combined using a weighted average. That is, weights may be accorded to each physiological parameter and the normalized values may be combined to determine a sedation level. In such an example, the sedation level may be normalized on the same scale as the constituent physiological parameter inputs. For example, a tidal volume, a respiratory rate, and a movement measurement may be generated. Each of these parameters may be normalized based on comparison to normal or expected ranges. Thus, each of these parameters may be normalized. Then, the normalized parameters may be combined to determine a sedation level. For example, a certain movement measurement may be determined (e.g., through clinical studies, machine learning, theoretical models, etc.) to be a stronger indication of a patient's sedation level than tidal volume or respiratory rate. In such an example, the normalized movement measurement may be given a higher weight in the combination of the normalized parameters, so that changes to the normalized movement parameter have a greater effect on the determined sedation level than do the other parameters. It will be appreciated, however, that any number of combination techniques are available. In other examples, a sedation level is determined based on identified patterns in breathing behavior, which may be reflected in breathing parameters. For instance, a breathing parameter may include an evaluation of whether a patient is experiencing periodic breathing (i.e., clusters of small breaths separated by intervals of apnea or near-apnea. In such an instance, a breathing parameter may be a numerical or non-numerical indication that a patient is experiencing periodic breathing. In an example, such an indication of periodic breathing may be used to determine that a patient is over-sedated.

In some examples, a combination of physiological parameters may be based on existing sedation-assessment scales, such as the RAS scale or the SAS scale previously mentioned. In such examples, the physiological parameters, either as measured and/or normalized, may be used to determine where the patient might fall on the existing assessment scales, and the sedation level may be provided in the format used by the existing assessment scales. For example, the sedation level may be designed to correspond to the RAS scale and sedation level may be based on a comparison of the generated physiological parameters to certain aspects of the RAS scale. As an example, a “−3” on the RAS scale corresponds to patient movement (but no eye contact) in response to voice stimulus. Thus, operation 608 may utilize an activity parameter that measures a patient's response to voice stimulus (e.g., a stimulus provided at operation 602). One activity parameter may correspond to a patient's eye movement in response to a stimulus, while another activity parameter may correspond to the patient's head and/or body movement in response to the stimulus. Values for both activity parameters may be generated utilizing the non-contact monitoring methods described herein. Thus, at operation 608, these activity parameters may be analyzed, and based on the analysis, a determination may be made that the patient's body moved in response to an auditory stimulus, but that the patient's eyes did not. In this example, operation 608 may determine that the physiological behavior corresponds with the description of RAS score “−3”, and the resulting sedation level may indicate as much.

In some examples, data from a ventilator are also used to determine a sedation level. For example, a ventilator (e.g., ventilator 400) may be operatively connected to a patient (e.g., patient 112) so as to provide breathing support in any number of manners. As discussed herein, intubated patients receiving breathing support from a ventilator are often at least partially sedated, and ensuring that the patient is properly sedated has many benefits. The ventilator may include one or more sensors (e.g., sensor 409). These sensors may be configured to measure and collect a variety of patient information. In an example, the ventilator sensors are configured to monitor pressure and flow of gas at a variety of points in the ventilation circuit. For example, pressure and flow sensors may be placed so as to model the patient's breathing behavior and optimize the ventilator's operation to provide safe and effective ventilation. The information collected from these sensors (hereinafter “sensor data”) may be received from the ventilator (e.g., using a wired connection or a wireless connection) at operation 608. The sensor data may be received by a computing device (e.g., computing device 200) and/or a server (e.g., server 225). The sensor data may be measured data from the sensors themselves, such as pressure and flow.

The sensor data may also be used to generate derived values, such as tidal volume, respiratory rate, minute volume, etc. In addition, the sensor data may be utilized with triggering and cycling algorithms to determine when a patient is attempting to breathe and, in some examples, the strength of the attempt of the patient. For example, spontaneous modes allow a spontaneously breathing patient to trigger inspiration during ventilation. In a spontaneous mode of ventilation, the ventilator begins (triggers) inspiration upon the detection of patient demand or patient effort to inhale. The ventilator ends inspiration and begins expiration (cycles to expiration) when a threshold is met or when a patient demand or effort for exhalation is detected. The patient effort and magnitude of the patient effort may be recorded for the patient. Collectively, the sensor data and the values derived from the sensor data, including patient effort characteristics, are referred to herein as ventilator data. The ventilator data may be utilized at operation 608 to determine a sedation level of the patient. This data may be used to assess the appropriateness of ventilator settings as well as appropriateness of sedation level.

In an example, a generated physiological parameter is a breathing parameter. In this example, the ventilator data may include data relating to the breathing parameter. For instance, the ventilator data may include a respiratory rate. In such an instance, the ventilator data may be used to confirm the accuracy of the breathing parameter generated from the received visual data feed. If the generated breathing parameter and the ventilator data are consistent, method 600 may continue to operation 610. If the generated breathing parameter is not consistent with the ventilator data, the method 600 may return to operation 604 for to receive new patient images. In other examples, the method 600 may continue to generate a sedation level, but may underweight the inconsistent generated breathing parameter in the sedation level determination. In still other examples, the method 600 may determine that the ventilator data is more likely to be accurate and may use a breathing parameter generated from the ventilator data in addition to or instead of the breathing parameter generated from the received images. The comparison of the two sources of data may also be used to determine that the patient effort is not resulting in a triggered breath from the ventilator.

In other examples, the generated physiological parameter is an activity parameter. In this example, the ventilator data may include data relating to the patient's breathing behavior, such as the frequency and/or strength of the patient's breathing effort. Thus, the ventilator data and the physiological parameter, used together, may provide information about both the patient's breathing behavior and the patient's activity levels, both of which may be indications of the patient's sedation level. In such an example, operation 608 may utilize both the ventilator data and the generated activity parameter to determine a sedation level.

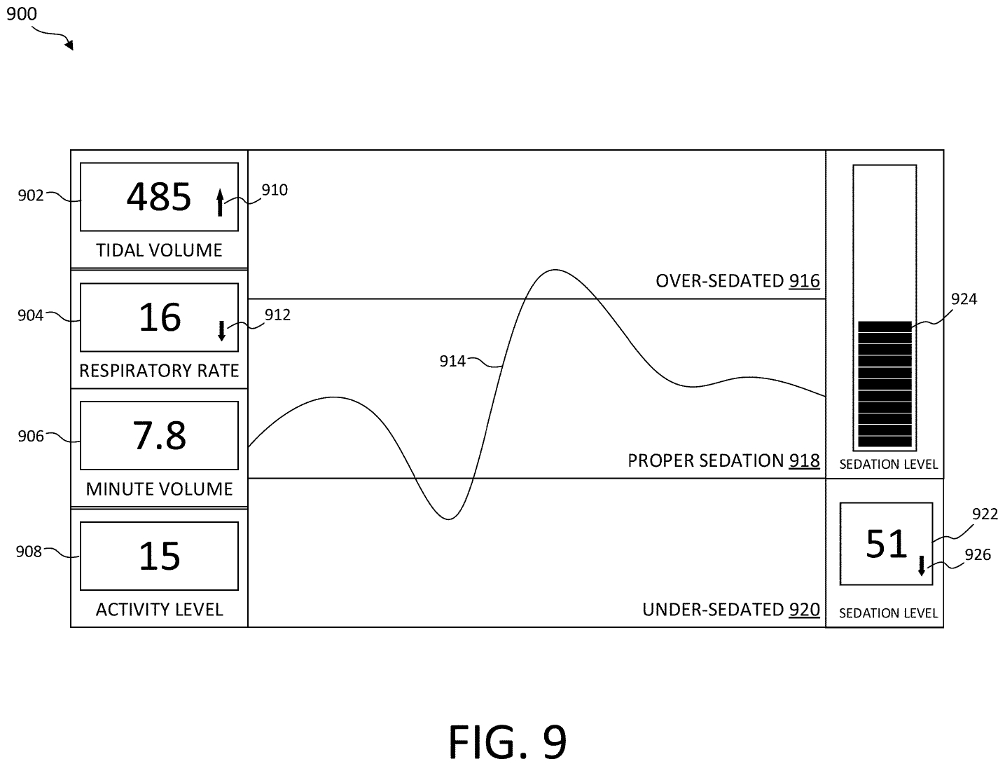

At operation 610, the sedation level of the patient is displayed. The sedation level may be displayed using a display similar to that depicted in FIG. 9. The sedation level may be displayed as part of any display associated with another device being used in the treatment of the patient. For instance, a ventilator used to ventilate the patient (e.g., ventilator 400) may include a display interface, and the sedation level may be displayed on this interface. In other examples, the sedation level may be displayed remotely from the patient. That is, the sedation level may be displayed at a centralized monitoring station at which care providers are able to monitor multiple patients simultaneously. The sedation level may also be displayed in a variety of forms. As depicted in FIG. 9, the sedation level for a patient may be displayed over a range of time (e.g., indicator 914). Additionally or alternatively, the sedation level may be displayed as an instantaneous or substantially real-time measure (e.g., sedation indicator 924 and/or sedation value 922). In FIG. 9, an example measure is displayed as a sedation indicator 924 with multiple levels, wherein the illumination of the levels of the bar indicates the patient's sedation level. Additional features of the display in FIG. 9 are discussed further below. It will be appreciated, however, that the sedation level can be displayed in any number of forms. For instance, the sedation level may be displayed as a colored indicator, where colors correspond to sedation levels. The sedation level may be displayed as a normalized numerical sedation value 922. A sedation level trend 926 may also be displayed. A sedation level trend may reflect the magnitude and the direction of change of the sedation level over a period of time. Any display form may be used that is capable of communicating the patient's sedation level to a human.

Returning to the method 600 in FIG. 6, at operation 612, a determination is made as to whether the sedation level is outside of a normal or expected range. This determination may be made by comparing the generated physiological parameter and/or the determined sedation level to one or more parameter and/or sedation thresholds. In an example, there is an upper threshold and a lower threshold for the sedation level. The normal or expected range for the sedation level falls between the lower and upper thresholds. Thus, if the sedation level exceeds the upper threshold or falls below the lower threshold, a determination may be made that the sedation level is outside the normal or expected range. The normal or expected range may be dependent on the physical characteristics of the patient, such as the patient's age, height, weight, gender, etc. The range may be dependent on the patient's health condition or treatment status, such as whether the patient is suffering from an illness or whether the patient has been sedated and/or for how long the patient has been sedated. Accordingly, when the patient's sedation level approaches or passes an upper or lower threshold, a determination may be made that the patient is over- or under-sedated.

If the determination made in operation 612 is “NO” (i.e., that the sedation level is not outside of the normal or expected range), the method 600 returns to operation 604 where the method 600 continues to receive images of the patient and continues to determine a sedation level for the patient. In this way, the method advantageously achieves continuous or substantially continuous monitoring of the patient, such that any deviation from the normal or expected range of sedation level will be quickly detected and an indication quickly provided.

If the determination made at operation 612 is “YES” (i.e., that the sedation level is outside of the normal or expected range), the method 600 may proceed to perform any or all of operations 614, 616, and 618. In one example, the method proceeds to operation 614, where an alarm is activated. Activating an alarm may include displaying an indicator on a display (e.g., the display of FIG. 9) indicating that the patient is over- or under-sedated. In another example, activating an alarm includes causing an audible tone or alarm to be sounded. Such an auditory alarm may be produced by the monitoring system described herein or may be produced by a different device, such as a device associated with a centralized patient-monitoring system. Alternatively or in addition, if the determination in operation 612 is “YES,” the method may proceed to operation 616 where an amount of sedative to be administered is determined. In an example, the amount of sedative may be based on the patient's sedation level and/or a rate of change of the patient's sedation level. For instance, the patient's sedation level may be approaching or may have exceeded a sedation threshold representing the upper limit of the normal or expected range of sedation levels for a patient. Based on the change to the patient's sedation level, a certain amount of sedative that is likely to return the patient to a normal sedation level may be determined. Additionally or alternatively, the determination can be made based on information about the patient. In an example, the determination is made at least in part based on one or more physical characteristics of the patient. For instance, the patient's age, gender, height, weight, medical history, etc. may affect how the patient responds to an administered sedative. Accordingly, the amount of sedative to be administered may vary depending on the patient's physical characteristics.

In some examples, the patient-monitoring system described herein may include or may be in communication with a device or devices capable of administering sedatives or any other pharmaceutical compound to the patient. In such examples, the method 600 may proceed to operation 618, where method 600 causes an amount of sedative to be administered. Causing an amount of sedative to be administered may involve sending a signal to a device capable of administering the sedative, such as a titration device. The signal may include the amount of sedative to be administered, or include a signal based on the amount of sedative to be administered. For instance, in an analog signal, the voltage level and duration of the signal may correspond to the determined amount of sedative to be delivered. In a digital signal, the bits of the signal may correspond to the determined amount of sedative to be delivered. In another example, the signal includes the timing of the sedative administration. For example, a determination may be made at operation 616 that a sedative should be administered over a period of time to safely return the patient to a normal sedation level. Alternatively, causing a sedative to be administered may include sending an alert to a care provider that a certain amount of sedative should be administered to return the patient to a normal sedation level. The care provider may be capable of administering the sedative in accordance with the instructions contained in the alert. In any of these examples, operations 616 and 618 may involve using a proportional-integral-derivative (PID) controller and/or other feedback loop devices or functions (e.g., fuzzy logic). In this example, the PID controller, and/or another feedback loop device, may continuously monitor the patient's sedation level and/or other physiological parameters and may continuously determine an amount of sedative to be administered based on analysis of the sedation level and/or physiological parameters.

FIG. 7 illustrates an example method 700 for patient monitoring. Method 700 begins at operation 702, where a visual data feed is received. All or a subset of the steps of method 700 may be executed by various components of a non-contact patient monitoring system, such as a local computing device (e.g., computing device 200), a server (e.g., server 225), another remote device, a display (e.g., display 422), an image capture device (e.g., camera 114), and/or other devices described herein. In an example, a visual data feed is a video feed captured by an image-capture device (e.g., image-capture device 285). In another example a visual data feed is a series of still images. Whether a video feed, a series of still images, or any other visual data feed, the received data feed may include image properties, such as those discussed herein with respect to the two or more captured images (e.g., image depth, temperature, color, brightness, movement, etc.).

At operation 704, the visual data feed is divided into segments corresponding to a plurality of time intervals. As an example, a visual data feed may be captured by an image-capture device (e.g., image-capture device 285). The visual data feed may be captured continuously or intermittently. In either instance, the constituent data in the visual data feed corresponds to a point in time. Accordingly, the visual data feed may be segmented according to the corresponding point in time. For example, a visual data feed may be divided into segments of a constant time interval (e.g., 1 second intervals, 10 second intervals, 1 minute intervals, etc.). In an example, the time interval may vary according to the patient's behavior. For instance, the time interval may be adjusted to correspond with the patient's breathing behavior, such that each segment of the visual data feed corresponds as closely as possible to a single breath cycle (i.e., an inhalation phase and an exhalation phase) of the patient. Thus, if the patient's respiratory rate increases, the segments of the visual data feed may become shorter as the patient's breathing cycle becomes shorter.

At operation 706, a physiological parameter is generated for each visual data feed segment. In the example described above where the segments correspond to a single breath cycle, a physiological parameter may be generated for each segment. For instance, for a single breath cycle, a tidal volume may be generated. As discussed herein, generating a physiological parameter may include detecting and analyzing changes in one or more image properties over a period of time. For example, for a single visual data feed segment, generating a physiological parameter may include defining an ROI of the patient's chest and detecting changes in the depth of the ROI in order to determine the change in the volume of the patient's chest over the course of one or more breath cycles. In another example, for a visual data feed segment, an amount of patient movement may be detected to generate an activity parameter. At operation 706, a physiological parameter is generated for two or more visual data feed segments. The physiological parameter for the two or more visual data feed segments may be the same physiological parameter or a different parameter. For each of the two or more visual data feed segments, multiple physiological parameters may be generated.

At operation 708, a generated physiological parameter for one of the two or more visual data feed segments is compared to a generated physiological parameter for a different one of the two or more visual data feed segments. For example, where the visual data feed segments correspond to a time interval of the patient monitoring, a physiological parameter for one time interval may be compared to the physiological parameter in the immediately preceding time interval. In another example, a physiological parameter over multiple visual data feed segments may be averaged before the physiological parameter is compared to a physiological parameter over one or more visual data feed segments. For instance, the physiological parameter for the most recent time interval may be compared to the patient's average physiological parameter over a longer period of time. For example, a patient's tidal volume for the patient's most recent breath may be compared to the patient's average tidal volume over the patient's last twenty breaths. It will be appreciated that, in addition to the examples provided above, there are many techniques for comparing physiological parameters across two or more visual data feed segments.

At operation 710, a trend is determined for a physiological parameter. As used herein, a trend for a physiological parameter may be any value or indication relating to a change—or lack thereof—in one or more physiological parameters over time. As an example, a trend may be a rate of change in a physiological parameter. Such a trend may be determined by comparing a physiological parameter between the most recently captured visual data feed segment and one or more previously captured segments, as described above with respect to operation 708. In another example, a trend may represent a longer-term measure of a patient's physiological parameter over time. In such an example, a trend may include a moving average of a physiological parameter. In another example, such a trend may include a direction or magnitude of one or more physiological parameters over a period of time.

At operation 714, a determination is made regarding whether the physiological parameter trend is outside a normal or expected range. In examples, the physiological parameter trend is a rate of change of a physiological parameter. A rate of change may be an instantaneous rate of change or may represent an amount of change over a unit period of time. In either case, a determination may be made that certain changes in a physiological parameter may be expected or normal. For instance, a determination may be made that a sedated patient's respiratory rate is normally within a certain range of values (as discussed previously) and that the respiratory rate is expected to fluctuate within that range of normal values. However, a determination may be made (e.g., through clinical studies, theoretical models, machine-learning results, etc.) that sudden fluctuation in respiratory rate is an indication that the patient is over- or under-sedated.

For instance, a determination may be made that a rapid increase or decrease in respiratory rate indicates over- or under-sedation, even when the respiratory rate itself remains within a normal or expected range for that physiological parameter. Thus, in order to monitor physiological parameter trends, a physiological parameter trend may be compared to one or more physiological parameter trend thresholds. In an example, each physiological parameter trend has a corresponding upper and lower trend threshold, and comparison to these thresholds allows determination of whether the trends are within the normal or expected range of trends. In this way, monitoring physiological parameter trends may allow earlier detection of over- or under-sedation by analyzing changes in physiological parameters and comparing those changes to normal or expected changes. It will be appreciated that the examples provided relating to respiratory rate are meant to be non-limiting and that the determination may be made with respect to any physiological parameter trend.

If the determination made at operation 714 is “NO” (i.e., that the physiological parameter is not outside a normal or expected range), method 700 returns to operation 702, where a visual data feed is received. From operation 702, monitoring may continue. Intermittent and/or continuous determination of physiological parameter trends may continue, and for each determined trend a determination made regarding whether the trend is outside a normal or expected range.

If the determination made at operation 714 is “YES” (i.e., that the physiological parameter is not outside a normal or expected range), method 700 proceeds to operation 716, where an indication is provided that the patient is under-sedated or over-sedated.

At operation 712, a physiological parameter trend is displayed. As with the physiological parameter or a sedation level, a physiological parameter trend may be displayed in any of many suitable forms. An example of an indication of a physiological parameter trend is provided as trend indicator 910 and trend indicator 912 in FIG. 9. In this non-limiting example, the size and direction of the arrow in indicators 910 and 912 represents the direction and magnitude of a physiological parameter trend. For example, trend indicator 910 is larger than trend indicator 912 and indicates an upward trend, while trend indicator 912 indicates a downward trend. In such an example, the indicators communicate that the patient's tidal volume is increasing, that the patient's respiratory rate is decreasing, and that the tidal volume is increasing more quickly than the respiratory rate is decreasing. Because, in some examples, the units of the physiological parameters may be different (e.g., tidal volume in mL and respiratory rate in breaths per minute), the trend may be normalized to adjust for this unit difference, so that the trend indicates the relative rate of change of the physiological parameter (e.g., a percent increase or decrease over a unit time). In this manner, a provider, patient, or other individual may be able to quickly perceive not only a physiological parameter, but also a corresponding trend for that parameter.

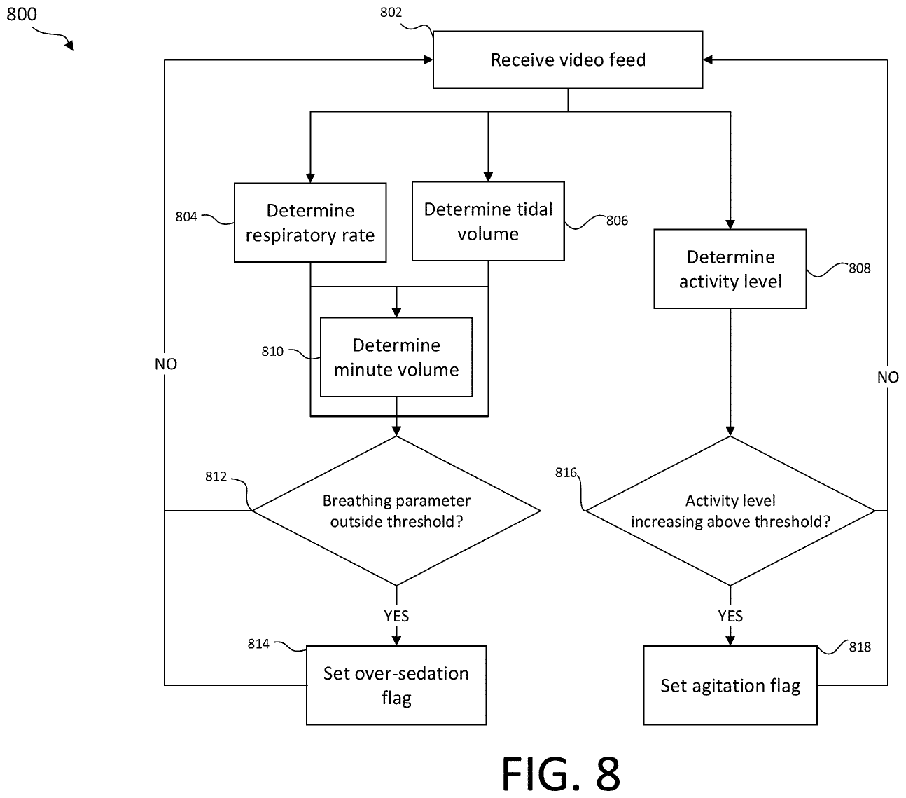

FIG. 8 illustrates an example method 800 for patient monitoring. Method 800 begins at operation 802, where a visual data feed in the form of a video feed is received. All or a subset of the steps of method 800 may be executed by various components of a non-contact patient monitoring system, such as a local computing device (e.g., computing device 200), a server (e.g., server 225), another remote device, a display (e.g., display 422), an image capture device (e.g., camera 114), and/or other devices described herein. In an example, the video feed is captured by an image-capture device (e.g., image-capture device 285) such as depth-sensing camera. The received video data feed may include image properties, such as those discussed herein with respect to the two or more captured images (e.g., image depth, temperature, color, brightness, movement, etc.).

Method 800 proceeds to one or more of operation 804, operation 806, and/or operation 808. At operation 804, a respiratory rate is determined based on the received video feed. Determining a respiratory rate may include using any of the techniques discussed herein, such as those discussed with respect to operation 508, operation 606, and operation 706. In an example, determining a respiratory rate involves analyzing information in the video feed captured from a depth camera. In such an example, a patient ROI corresponding to a patient's chest may be identified and changes in depth of the patient ROI analyzed to determine that a patient has a certain respiratory rate. Respiratory rate may be based on a substantially immediate analysis of a video feed or, alternatively, may be based on a video feed over a period of time. For example, respiratory rate may indicate the number of breaths the patient took in the past minute. In other examples, respiratory rate may indicate a time-weighted average of the patient's breathing rate.

At operation 806, a tidal volume is determined based on the received video feed.

Determining a tidal volume may advantageously include using any of the techniques discussed herein, such as those discussed with respect to operation 508, operation 606, and operation 706. In an example, determining a tidal volume involves analyzing information from a depth camera. In such an example, a patient ROI corresponding to a patient's chest may be identified and changes in depth of the patient ROI analyzed to determine that a patient has a certain tidal volume. For example, a change in depth may be multiplied by an estimated and/or measured lung capacity to determine that a change in patient ROI depth indicates that a certain amount of gas has been inhaled/exhaled by the patient. Tidal volume may be based on a substantially immediate analysis of a visual data feed or, alternatively, may be based on a visual data feed over a period of time, such as an average of tidal volume over a number of patient breaths.

At operation 810, a patient's minute volume is determined based on the video data feed and/or the determined respiratory rate and tidal volume. Determining a minute volume may include using any of the techniques discussed herein, such as those discussed with respect to operation 508, operation 606, and operation 706. Determining a minute volume may include analyzing information from the video feed captured with a depth camera and determining changes in volume over time. Alternatively, minute volume may be determined from analysis of parameters already determined. Minute volume is a measure of the volume of gas inhaled or exhaled from a person's lungs per minute. Accordingly, one technique for determining minute volume may be to multiply a patient's tidal volume by the patient's respiratory rate (if measured on a per-minute basis). As discussed herein, tidal volume and respiratory rate for a patient may be real-time measurements of a patient's breathing behavior or may be based on data over a period of time. In either example, minute volume may be determined by some analysis (e.g., multiplication) of these values.

At operation 812, a determination is made regarding whether one or more of the breathing parameters determined at operations 804, 806, and/or 810 fall(s) outside of a normal or expected range for the parameter(s). Determining whether a breathing parameter falls outside of a normal or expected range for the parameter may include using any of the techniques discussed herein, such as those discussed with respect to operation 510, operation 612, and operation 714. In an example, operation 812 determines whether one or more of the breathing parameters falls below a parameter threshold. In such an example, a lower threshold may be used because lower breathing parameters (e.g., respiratory rate) correspond to higher levels of sedation. In this example, breathing parameters under a lower threshold may indicate a patient is over sedated. At operation 812, any one or more of tidal volume, respiratory rate, and minute volume may be used to assess a patient's breathing, and any one or more of these breathing parameters may be compared to a lower threshold. In examples where more than one of the parameters are compared to a parameter threshold, operation 812 may further involve determining how many of the parameters falls outside of a normal range and, if so, by how much the parameter falls outside the range. Any of this information may be utilized in determining whether breathing parameters fall outside the normal or expected range. For example, the determination in operation 812 may be based on the value of the breathing parameter and the rate of change of the breathing parameter. As an example, if the breathing parameter is above but near (e.g., within a tolerance such as 10%) a lower threshold and decreasing, the determination in operation 812 may be that that the breathing parameter is outside the parameter threshold (i.e., “YES”). In contrast, if the breathing parameter is below but near (e.g., within a tolerance such as 10%) a lower threshold and increasing, the determination in operation 812 may be that the parameter is within the threshold (i.e., “NO”). Accordingly, over-sedation may be identified more quickly, but false identifications of over-sedation may also be avoided.

If the determination at operation 812 is “YES”, method 800 proceeds to operation 814, where an over-sedation flag is set. As will be appreciated in the context of computing, a flag may be a variable whose value indicates the attainment of some designated state or condition by an item of equipment or program. The flag may then be subsequently used as a basis for conditional branching and similar decision processes. For example, as used in the present context, the over-sedation flag may be any value and/or signal (e.g., a binary true/false signal) stored as an indication of whether the patient is over-sedated based on the determination made in operation 812. For instance, a variable corresponding to the over-sedation flag may be a 1 or a 0 stored in any suitable computer-readable storage media. A value of 1 (or high) may correspond to over-sedation.

The over-sedation flag may be read or queried by other components of a program or computer-implemented method, such as those discussed herein. Decisions may then be made based on the whether the flag is set or not (e.g., whether the corresponding variable is high or low). Setting of the over-sedation flag (e.g., changing the corresponding variable to high) may cause or trigger other actions to be performed. In an example, a visual or auditory indication of over-sedation may be generated upon the over-sedation flag being set in operation 814. In some examples, setting of the over-sedation flag may not cause any indication of over- or under-sedation until a query is made regarding the patient's sedation level. In an example, a titration device may be configured to make periodic queries regarding the patient's sedation level. If the over-sedation flag is set, the titration device may cease the delivery of sedative to the patient until the flag is removed (e.g., the corresponding variable is set to low).

As should be appreciated based on the foregoing, an under-sedation flag may also be set if a determination is made in operation 812 that the breathing parameters are increasing outside the parameter threshold. If an under-sedation flag is set to indicate under-sedation, a query by a titration device may return an indication that the patient is under-sedated, and the under-sedation flag may cause the titration device to administer an amount of sedative. In some examples, sedative is administered until the under-sedation flag is removed (e.g., the corresponding variable is set to low).

If the determination at operation 812 is “NO”, method 800 returns to operation 802, and monitoring continues with receiving the video data feed. If an over-sedation flag had been previously set, the over-sedation flag may be removed upon a determination of “NO” at operation 812.

Returning to operation 802, method 800 may additionally or alternatively proceed from operation 802 to operation 808. At operation 808, an activity level is determined based on the video feed received in operation 802. Determining an activity level may include using any of the techniques discussed herein, such as those discussed with respect to operation 508, operation 606, and operation 706. In an example, determining an activity level involves analyzing information from a depth camera. In such an example, a patient ROI corresponding to a patient's chest may be identified and changes in depth of the patient ROI analyzed to determine that a patient has a certain activity level. For example, a patient ROI corresponding to a patient's head, neck, or limbs may be identified. In such an example, changes in depth and/or movement of the patient ROI may be analyzed to determine that a patient is moving a certain amount or is moving in a certain way (e.g., head movement). An activity level may be based on a substantially immediate analysis of a visual data feed or, alternatively, may be based on a visual data feed over a period of time, such as frequency of patient movement.

At operation 816, a determination is made regarding whether the determined activity level is outside of a normal or expected range. Determining whether a breathing parameter falls outside of a normal or expected range for the parameter may include using any of the techniques discussed herein, such as those discussed with respect to operation 510, operation 612, and operation 714. In an example, operation 816 determines whether the activity level exceeds an upper threshold. In such an instance, a determination may be made that no activity or a low level of activity is consistent with proper sedation and therefore is less helpful in determining a patient's sedation or agitation level. In this instance, the activity level may be compared to an upper activity-level threshold so as to determine whether patient activity (e.g., movement) is higher than would be expected of a properly sedated patient.

If the determination at operation 816 is “YES”, method 800 proceeds to operation 814, where an agitation flag is set. In some examples, agitation corresponds to under-sedation. In other examples, agitation may be caused by increasing or decreasing (i.e., waxing or waning) sedation. The agitation flag may therefore be associated with the sedation level and may be used in determining a sedation level. Alternatively, agitation may be determined separate from the sedation level and each measure may be indicated to a care provider separately. The agitation flag may be similar to the over-sedation flag discussed above in that the agitation flag may be a variable that indicates whether the patient is agitated. Setting of the agitation flag may be an indicator that the patient is under-sedated or has received too much sedation for too long. Setting of the agitation flag may cause a visual indicator to be displayed and/or an audible alarm to be sounded. In some examples, if the agitation flag is set, a query by a titration device may cause the titration device to administer an amount of sedative. In some examples, sedative is administered until the agitation flag is removed (e.g., the corresponding variable is set to low). Once the agitations flag is set in operation 818, the method 800 returns to operation 802, and monitoring continues with receiving a video feed.

If the determination at operation 816 is “NO”, method 800 returns to operation 802, and monitoring continues with receiving a video feed. If an agitation flag had been previously set, the agitation flag may be removed upon a determination of “NO” at operation 812.

FIG. 9 illustrates an example display 900. In this non-limiting example, the display 900 provides indications of a variety of physiological parameters, such as tidal volume 902, respiratory rate 904, minute volume 906, and activity level 908. In this example, these physiological parameters 902-908 are displayed as numerical values. These numerical values may represent a physical unit of measurement (e.g., the tidal volume may represent the mL of air inhaled and exhaled). Alternatively, the numerical values may be normalized according to expected values for a patient. For example, the tidal volume may be a value between 1 and 100, where 50 represents the expected tidal volume and values below or above 50 represent deviations from the expected tidal volume for a given patient. It will be appreciated, however, that the display may not include numerical values.

In other examples, the display may include a graph charting the physiological parameters over time. In other examples, the display may include an indicator representing a trend for a physiological parameter, such as trend indicators 910 and 912 indicating the trends for tidal volume and respiratory rate, respectively. In such an example, the displayed trend may represent the direction of change of the physiological parameter over a given time period. The displayed trend may also represent the magnitude of change of the physiological parameter over that time period. For instance, the size of the indicator may represent the magnitude of the rate of change. Any of these indicators may additionally include color-coding. In such an example, the colors of the indicators may communicate something about the physiological parameter to a care provider. For instance, the color of a numerical value may indicate that the physiological parameter is outside of a normal range.

Display 900 also provides an indication of the patient's sedation level over a period of time. In this example, display 900 charts the patient's sedation level as a signal over time 914. The display 900 includes three sedation regions: an over-sedation region 916, a proper sedation region 918, and an under-sedation region 920. It will be appreciated, however, that the sedation level may be depicted without these regions. It will also be appreciated that the sedation level may be displayed in a variety of other forms. For instance, the sedation level may be a numerical value, such as the numerical sedation value 922. In other examples, the sedation level may be indicated by a colored indicator. In this example, certain colors may correspond to different sedation levels, such that the color of the indicator communicates the patient's sedation level to a care provider. In display 900, sedation level is further indicated by sedation indicator 924. In this example, sedation level corresponds to the level of the sedation indicator 924. As sedation level increases, sedation indicator 924 rises. As sedation level decreases, sedation indicator 924 falls. In examples, display 900 may also contain a sedation level trend 926. In such an example, the displayed trend may represent the direction of change of the patient's sedation level over a given time period. The displayed trend may also represent the magnitude of change of the patient's sedation level over that time period. For instance, the size of the indicator may represent the magnitude of the rate of change.

It will be appreciated that display 900 may contain any other number of indicators that may be relevant to and/or useful in monitoring a patient's health. It will further be appreciated that display 900 may be displayed on a standalone display device (e.g., display 122). Additionally or alternatively, display 900 may be displayed as part of some other integrated monitoring device. For example, display 900 may be included on a screen or other display of a ventilator (e.g., display 422 of ventilator 400). In other examples, display 900 may be shown as part of a centralized monitoring station, where a care provider may be able to view or access similar displays for a variety of patients substantially concurrently.

Those skilled in the art will recognize that the methods and systems of the present disclosure may be implemented in many manners and as such are not to be limited by the foregoing exemplary aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein.

In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with many systems such as ventilators for non-human patients, invasive or non-invasive ventilation, and other gas transport systems, and various types of event detection.

What is claimed is:

1. A computer-implemented method for patient monitoring, the method comprising:
receiving, from an image-capture device, two or more images of a patient, wherein the two or more images include an image property;
based on the received two or more images, determining, by a computing device, a physiological parameter of the patient, wherein the physiological parameter is at least one of a breathing parameter or an activity parameter;
comparing, by the computing device, the physiological parameter to a parameter threshold;
based on comparing the physiological parameter to the threshold, determining, by the computing device, a sedation level of the patient;
based on the determined sedation level, performing at least one of:
administering, by a titration device, a sedative;
displaying an indication of the determined sedation level; or
activating an alarm.

2. The method of claim 1, wherein the image-capture device is a depth camera and wherein the image property is image depth.

3. The method of claim 1, wherein displaying the indication of the sedation level includes displaying at least one of a numerical value for the sedation level or a signal over time for the sedation level.

4. The method of claim 1, wherein generating the physiological parameter comprises calculating a change between the image property in the two or more images.

5. The method of claim 1, wherein the breathing parameter is at least one of a tidal volume, a minute volume, or a respiratory rate.

6. The method of claim 1, wherein generating the physiological parameter comprises:
generating the physiological parameter from a first series of images;
generating the physiological parameter from a second series of images;
comparing the physiological parameter from the first series of images to the physiological parameter from the second series of images; and
based on comparing the physiological parameter from the first group of images to the physiological parameter from the second group of images, determining a trend for the physiological parameter.

7. The method of claim 6, wherein determining the trend comprises determining a rate of change of the physiological parameter.

8. The method of claim 1, wherein the physiological parameter is a first physiological parameter, wherein the threshold is a first threshold, and wherein the method further comprises:
generating a second physiological parameter from the two or more images;
comparing the second physiological parameter to a second threshold; and
wherein determining the sedation level is further based on the comparison of the second physiological parameter to the second threshold.

9. A system for patient monitoring, the system comprising:
an image-capture device configured to capture two or more images of a patient;
a display;
a processor;
memory, operatively connected to the processor, storing instructions that, when executed, cause the system to:
receive the two or more images from the image-capture device;
generate a physiological parameter from the two or more images, wherein the physiological parameter is one of a breathing parameter or an activity parameter;
determine a sedation level of the patient from the physiological parameter; and
based on the determined sedation level, perform at least one of:
displaying, on the display, an indication of the determined sedation level; or
activating an alarm.

10. The system of claim 9, wherein determining the sedation level comprises comparing the physiological parameter to a threshold, wherein the threshold is based on a physical characteristic of the patient, wherein the physical characteristic is one of patient height, patient weight, patient gender, or patient age.

11. The system of claim 9, wherein the system further comprises a titration device and the instructions, when executed, cause the titration device to administer an amount of sedative based on the determined sedation level.

12. The system of claim 9, wherein the physiological parameter is a breathing parameter, wherein the instructions further cause the processor to generate an activity parameter from the two or more images and wherein determining the sedation level of the patient comprises:
comparing the breathing parameter to a breathing parameter threshold;
comparing the activity parameter to an activity parameter threshold; and
determining the sedation level based on both comparisons.

13. The system of claim 12, wherein determining the sedation level comprises determining that at least one of:
the breathing parameter is below the breathing parameter threshold; or
the activity parameter is above the activity parameter threshold.

14. A computer-implemented method for patient monitoring, the method comprising:
receiving two or more images of a patient;
generating a plurality of physiological parameters based on the two or more images;
determining a sedation level based on the plurality of physiological parameters;
comparing the sedation level to a sedation threshold; and
based on determining that the sedation level falls below the sedation threshold, performing at least one of:
setting an under-sedation flag; or
causing an amount of a sedative to be administered to the patient.

15. The method of claim 14, wherein the amount of sedative is based on a physical characteristic of the patient and the determined sedation level.

16. The method of claim 14, wherein the two or more images have been captured from at least one of a depth camera, an infrared camera, or a red-green-blue (RGB) camera.

17. The method of claim 14, further comprising:
receiving ventilator data from a ventilator providing ventilation to the patient; and
wherein the determined sedation level is further based on the ventilator data.

18. The method of claim 14, further comprising activating a patient prompt prior to or concurrently with receiving the two or more images.

19. The method of claim 18, wherein the patient prompt is one of an auditory stimulus or a visual stimulus.

20. The method of claim 19, wherein receiving two or more images comprises receiving a first image captured before activation of the patient prompt and receiving a second image captured after activation of the patient prompt, and wherein generating the plurality of physiological parameters comprises comparing the first image to the second image.
